(19) United States
(12) Patent Application Publication
(10) Pub. No.: US 2024/0398443 A1
(43) Pub. Date: Dec. 5, 2024

(54) Kit and Method for Removing Tick from a Mammal

(71) Applicant: Mark Dennis Lenius, Brandon (CA)

(72) Inventor: Mark Dennis Lenius, Brandon (CA)

(21) Appl. No.: 18/325,045

(22) Filed: May 29, 2023

(51) Int. Cl. A61B 17/50

(52) U.S. Cl. CPC A61B 17/50; A61B 2017/505

(57) ABSTRACT

A kit for removing an imbedded tick from epidermal tissue of a mammal comprises a container configured to receive a liquid with a rim sized to surround the tick and a prescribed volume of liquid disinfectant in the container and adapted to sanitize the tissue and tick. The prescribed volume is less than a container volume to provide an air gap between the rim and a top surface of the liquid disinfectant so that the disinfectant can flow to fill the neck when the container is tilted. A method for tick extraction comprises providing the container with the prescribed volume of liquid disinfectant therein; locating the container rim on the mammal in a manner forming a seal therewith and surrounding the tick; submerging the tick in the liquid disinfectant for a prescribed duration; and removing, from the mammal, the container with the liquid disinfectant substantially received therein and the tick.

### FIELD OF THE INVENTION

The present invention relates to a kit and method for removing a tick from a mammal, and more particularly to such a kit and method comprising liquid disinfectant applied to epidermal tissue of the mammal.

### BACKGROUND

There are challenges in removing a tick, an animal belonging to mite superorder Parasitiformes and also commonly referred to wood ticks, after its attachment to epidermal tissue of a mammal. Conventional methods include contacting the tick with a hot implement or article to urge release or withdrawal from the epidermal tissue, and/or (forcibly) mechanically removing the tick from the epidermal tissue. After removal, the epidermal tissue at a location of a bite of the tick is sanitized to remove any tick-borne pathogens which may have transferred from the tick to the mammal.

### SUMMARY OF THE INVENTION

According to an aspect of the invention there is provided a kit for removing an imbedded tick from epidermal tissue of a mammal comprising:
a container configured to receive a liquid, wherein the container has a body and a neck collectively defining an interior of the container for receiving the liquid, wherein the body has a bottom arranged for resting on a support surface, wherein the neck has a rim delimiting an opening for transferring the liquid between the interior of the container and an exterior thereof, wherein the container further includes a cap configured to be removably received on the neck to selectively close the opening to resist transfer of the liquid from the interior to the exterior;
wherein the rim is arranged to be sized to surround the tick;
a prescribed volume of liquid disinfectant received in the container, wherein the liquid disinfectant is adapted to sanitize the epidermal tissue and the tick;
wherein the prescribed volume of liquid disinfectant is less than a volume of the interior of the container so as to provide an air gap between the rim of the container and a top surface of the liquid disinfectant in the container, such that the liquid disinfectant can flow to fill the neck upon tilting the container.

This provides a relatively simple arrangement for removing a tick which additionally acts to sterilize both a contact or bite area of the mammal and the tick.

Preferably, the kit further includes tweezers for mechanically removing the tick.

In one arrangement, the tweezers are configured to be received in the container in a storage position of the tweezers.

In one such arrangement, the tweezers are attached to the cap so as to be disposed in the storage position when the cap is received on the neck of the container.

In the illustrated arrangements, when the container extends between the bottom and the rim along an axis, the body of the container is enlarged relative to the neck in a radial direction of the axis and the neck is elongated relative to an axial direction of said axis.

In one arrangement, the body of the container is enlarged relative to the neck non-symmetrically around the axis such that the axis is offset from a center of the body.

Preferably, the neck has a height in the axial direction that is at least one-third of a height of the body in the axial direction.

In the illustrated arrangements, the height of the neck is at least equal to the height of the body in the axial direction.

Preferably, the liquid disinfectant is alcoholic and has an alcohol content of at least 70% by volume.

In the illustrated arrangements, the alcohol content is at least 90% by volume. Preferably, the alcohol content is 99% by volume.

Preferably, the rim has an inner diameter of about one-quarter inches to about one-half inches.

In the illustrated arrangements, the inner diameter of the rim is about ⅜ inches.

In one arrangement, the prescribed volume of the liquid disinfectant is greater than a volume of the neck of the container.

According to another aspect of the invention there is provided a method for extracting an imbedded tick from epidermal tissue of a mammal comprising:
providing a container with a prescribed volume of liquid disinfectant therein, wherein the prescribed volume of the liquid disinfectant is less than a volume of the container;
with the liquid disinfectant received in the container, locating a rim of the container on the mammal in a manner forming a seal therewith and surrounding the imbedded tick;
submerging the imbedded tick in the liquid disinfectant received in the container for a prescribed duration; and
removing, from the mammal, the container with the liquid disinfectant substantially received therein and the tick.

In one arrangement, removing the tick from the mammal comprises mechanically removing the tick using a tool distinct from the container.

In one arrangement, the prescribed duration for submerging the imbedded tick in the liquid disinfectant is at least 30 seconds.

### BRIEF DESCRIPTION OF THE DRAWINGS

The invention will now be described in conjunction with the accompanying drawings in which:

FIG. 1 is an elevational view schematically showing an arrangement of kit according to the present invention;

FIG. 2 is a top plan view schematically showing the arrangement of kit of FIG. 1 but without a cap of the container and tweezers;

FIG. 3 is an elevational view schematically showing another arrangement of kit according to the present invention;

FIG. 4 is a top plan view schematically showing the arrangement of kit of FIG. 3 but without the cap of the container and the tweezers;

FIG. 5 is a schematic illustration of a container of the kit applied to a mammal carrying an imbedded tick from a partial side view of the container; and

FIG. 6 is a schematic illustration of the container of the kit applied to the mammal carrying the imbedded tick, as in FIG. 5, but in plan view.

In the drawings like characters of reference indicate corresponding parts in the different figures.

### DETAILED DESCRIPTION

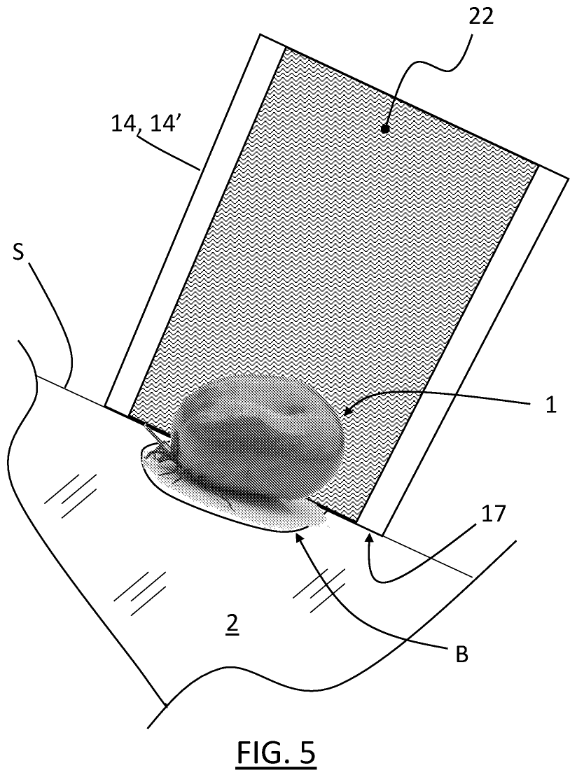

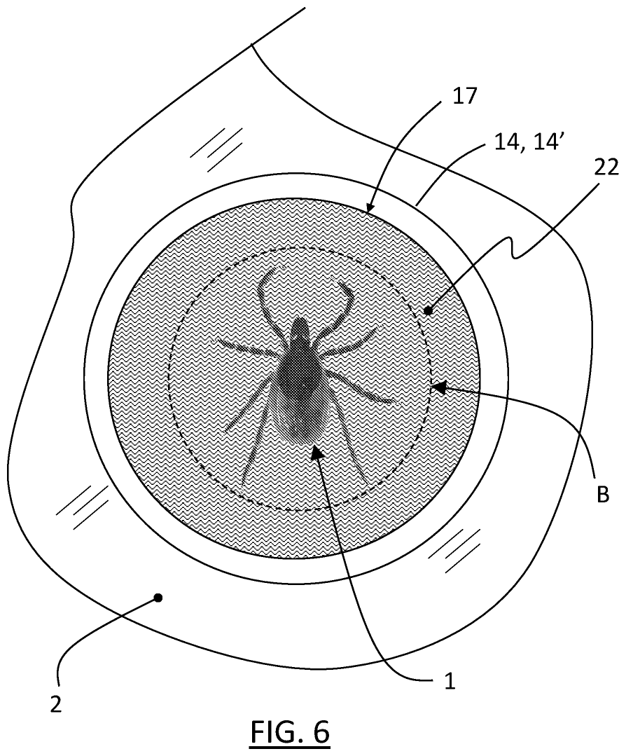

The accompanying figures show a kit, generally indicated at 10, for removing an imbedded tick 1 from epidermal tissue 2 of a mammal, more clearly shown in FIGS. 5 and 6. Since the kit 10 is suited for use to remove a tick which is imbedded, meaning the tick 2 is located at least partially beneath a surface S of the epidermal tissue or a skin of the mammal, removal using the kit may be considered extraction of the tick from the epidermal tissue or more generally from the mammal.

The removal kit 10 generally comprises a container 12 configured to receive a liquid, which has a body 13 and a neck 14 collectively defining an interior I of the container for receiving the liquid. The body 13 has a bottom 16 arranged for resting on a support surface, such as a surface of a tabletop, and the neck has a rim 17 delimiting an opening 17A for transferring the liquid between the interior of the container and an exterior thereof. The container 12 further includes a cap 19 configured to be removably received on the neck 14 over the rim 17, for example by being cooperatively threaded, to selectively close the opening 17A to resist transfer of the liquid (received in the container) from the interior to the exterior. The rim 17 is arranged to be sized to surround the tick, particularly in a diametric dimension, as will be better appreciated later.

The kit 10 further generally comprises a prescribed volume of liquid disinfectant 22 received in the container, which liquid disinfectant is adapted to sanitize the epidermal tissue 2 and the tick 1. The prescribed volume of liquid disinfectant is less than a volume of the container, that is the volume of the interior thereof which can receive liquid, so as to provide an air gap between the rim 17 of the container and a top surface 22A of the liquid disinfectant in the container, such that the liquid disinfectant can flow to fill the neck 14 upon tilting the container. That is, the prescribed volume of the liquid disinfectant is sized, relative to the interior volume of the container, such that in an upstanding orientation of the container, in which the rim 17 is above the bottom 16 and typically substantially vertically in-line therewith, the liquid disinfectant substantially fills a body portion of the container volume, that is a portion of the total volume of the container formed by the container body 13, and substantially does not fill the neck 14. Thus, the resultant air gap or unoccupied portion of the volume of the container is sized so that the liquid disinfectant can rapidly or violently gravitationally flow within the container when the container is tilted.

In the illustrated arrangements, the prescribed volume of the liquid disinfectant is greater than a volume of the neck of the container, that is a portion of the total container volume formed by the neck 14 between the rim 17 and a transition or joint 23 of the neck 14 and the body 13. Thus, when the container is tilted from the upstanding orientation, the neck of the container is full with the liquid disinfectant.

In the illustrated arrangements, the container 12 extends between the bottom 16 and the rim 17 along an axis A such that the container is symmetrical around the axis A. Furthermore, the body 13 of the container is enlarged relative to the neck 14 in a radial direction of the axis A, and the neck 14 is elongated relative to an axial direction of the container axis A. That is, the neck is taller in the height or axial direction than it is diametrically wide. More specifically, in the illustrated arrangement, the neck 14 has a height of at least one-third of a height of the body 13 in the axial direction. As such, the body 13 forms a majority of the volume of the container while occupying a minimal height in the axial direction of the axis A as compared to the neck. In the illustrated arrangement, the height of the neck is at least equal to the height of the body in the axial direction, and in some arrangements the neck is taller than the body.

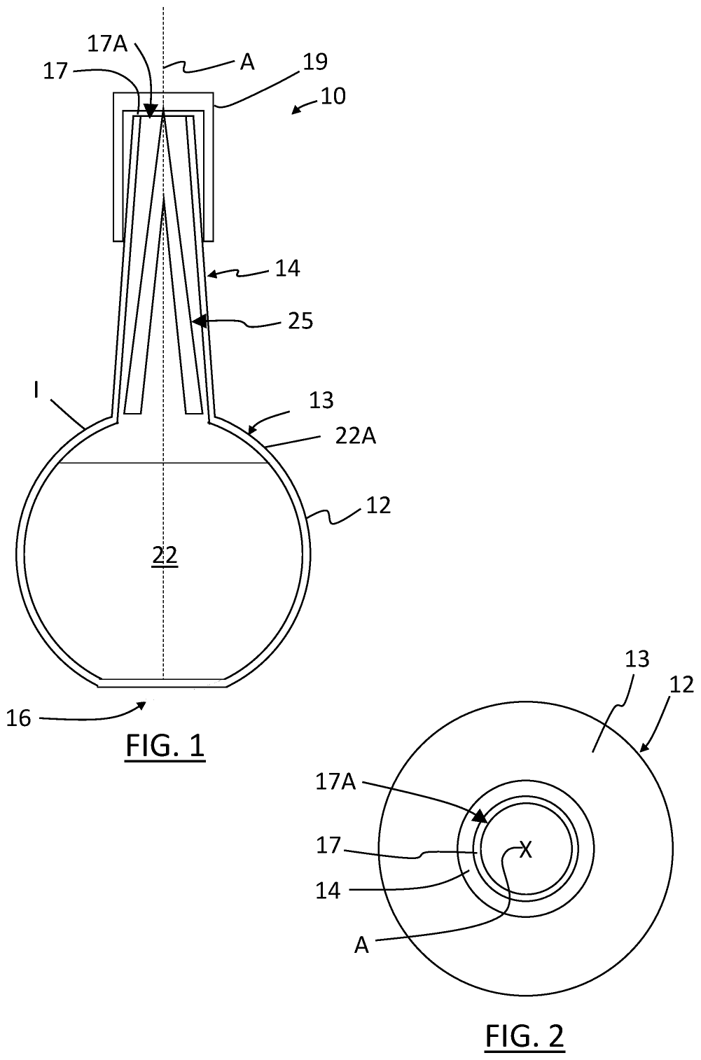

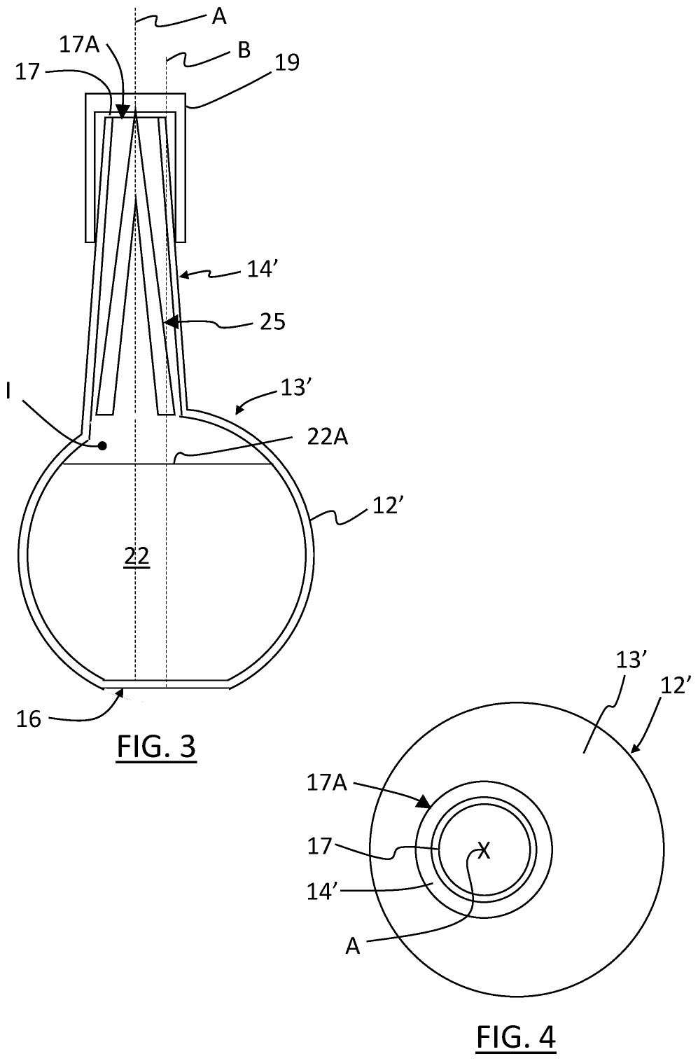

In a first one of the illustrated arrangements shown in FIGS. 1 and 2, the body 12 is enlarged relative to the neck 14 symmetrically around the axis A such that a center of the body is registered or coincident with the axis A. In a second one of the illustrated arrangements of FIGS. 3 and 4 having container 12′, body 12′ is enlarged relative to neck 14′ non-symmetrically around the axis A such that the axis is offset from a center of the body. As such, in the second arrangement, the neck 14′, which is formed symmetrically around the axis A, is offset relative to a vertically upstanding axis B passing through the body's center. Consequently, the container 12′ of the second arrangement may be tilted by a smaller degree or angle from an upstanding orientation, and in particular a vertically upstanding orientation when the axis A is vertical, and particularly when tilted from the axis B in a direction towards axis A, for filling or flooding the neck with the liquid disinfectant, as compared to the container 12 of the first arrangement. Besides the aforementioned features, the two illustrated arrangements are identical.

In further regard to the container, the rim 17 has a prescribed inner diameter of about one-quarter inches to about one-half inches, and preferably about ⅜ inches. This is considered satisfactory to encompass a variety of types of tick and varying size of the tick depending on a stage of feeding on the mammal, that is an amount of blood withdrawn by the tick from the mammal in which the tick is imbedded.

Turning now to the disinfectant 22, the liquid disinfectant is alcoholic and has an alcohol content of at least 70% by volume, and preferably of at least 90% by volume. This allows the disinfectant to act to sterilize the tick and the mammal's tissue more quickly. Preferably, the alcohol content is 99% by volume so as to urge release of the tick in a minimum amount of time. In the illustrated arrangement, the liquid disinfectant is isopropanol, alternatively referred to as isopropyl alcohol.

To complement the container 12 and the liquid disinfectant 22 received therein, the kit 10 preferably further includes tweezers 25 for mechanically removing the tick, for example if the mammal has significant hair or fur covering the epidermal tissue. Furthermore, the tweezers act as a device for physically or mechanically separating hair or fur of the mammal to facilitate visual inspection of the bite area to verify that the tick has released from the epidermal tissue. In the illustrated arrangement, the tweezers 25 are configured to be received in the container 12 in a storage position of the tweezers. Furthermore, in the illustrated arrangement the tweezers 25 are attached to the cap 19 so as to be disposed in the storage position when the cap is received on the neck 14 of the container to close the opening 17A. When the neck 14 is elongated, as in the illustrated arrangement, the tweezers 25 are sized to be substantially received in the neck, in other words to register with the neck in the storage position, and preferably not to extend into the body 13 of the container so as not to affect a height of the prescribed volume of the liquid disinfectant 22 in the container when the container is closed by the cap.

There is also disclosed herein a method for removing or extracting an imbedded tick 1 from epidermal tissue 2 of a mammal. The method generally comprises the steps of:
1) providing the container 12 with the prescribed volume of liquid disinfectant 22 therein;
2) with the liquid disinfectant 22 received in the container 12, locating a rim 17 of the container on the mammal in a manner forming a seal therewith and surrounding the imbedded tick;
3) submerging the imbedded tick 1 in the liquid disinfectant received in the container for a prescribed duration; and
4) removing, from the mammal, the container with the liquid disinfectant substantially received therein and the tick.

Typically, the container 12 is closed with a cap 19 so that the liquid disinfectant is retained therein.

It will be appreciated that the prescribed volume of the liquid disinfectant 22 is less than a volume of the container.

Sealingly locating the container rim 17 on the mammal as at step 2 above typically comprises pressing the rim 17 against the epidermal tissue surrounding the tick 1 and a bite area B on the epidermal tissue 2. Typically, in a human, the bite area B is visually denoted by redness in the epidermal tissue. Since the tick 1 is located at an epicenter of the bite area B, when the liquid disinfectant 22 is transferred to the neck portion of the interior of the container, the liquid disinfectant comes into contact with both the tick 1 and the epidermal tissue 2 and accordingly acts to sterilize both of same.

In the step of sealingly locating the container rim 17 on the mammal in a manner surrounding the imbedded tick, the container 12 is oriented in a manner such that the liquid disinfectant is not at the rim 17. Thus, spillage of the liquid disinfectant prior to formation of the seal on the mammal is substantially resisted. As such, in order to submerge the tick after the seal has been formed at an interface of the rim 17 and the epidermal tissue of the mammal, the container is tilted so that the bottom is above the rim 17, so as to be oriented in a downwardly facing condition. As such, the liquid disinfectant acts to fill or occupy an end of the neck portion including a full cross-sectional area of the neck at the rim.

Submerging the imbedded tick in the liquid disinfectant while the same is contained in the container 12 acts to shock the tick thereby urging the tick to disengage or release (its mouth) from the epidermal tissue of the mammal. Accordingly, the liquid disinfectant is adapted to chemically shock the tick.

Removing the container with the liquid disinfectant substantially received therein as at step 4 above means that the prescribed volume of the liquid disinfectant remains substantially the same before and after application to the tick (and the mammal). The container 12 acts to contain the liquid disinfectant 22 throughout the tick removal or extraction process, and when the liquid disinfectant is conveyed away from the bite area B after submersion for the prescribed duration, gravitational flow of the disinfectant in the container may act to disassociate the tick from the mammal and carry it away from same. In this manner, the container 12 may act to retain the liquid disinfectant and to contain the tick after removal from the mammal. Thus, after removal from the mammal, the tick may be handled via the container, so as to be handled indirectly, and disposed of by dumping to waste the disinfectant carrying the tick or the container in its entirety.

Optionally, removing the tick from the mammal comprises mechanically removing the tick using a tool distinct from the container, for example tweezers. This may be useful when the mammal has lots of hair in the area of the tick bite. Additionally or alternatively, this may act as an inspection tool to move or separate hairs in the area of the tick bite to visually inspect if the tick has released from the epidermal tissue.

The prescribed duration for submerging the imbedded tick in the liquid disinfectant is at least 30 seconds. Typically, a maximum of a range of prescribed duration is about 60 seconds. A specific length of time for submersion is dependent on a concentration of sanitizing or sterilizing agent in the disinfectant.

This provides a relatively simple arrangement for removing a tick which additionally acts to sterilize both a contact or bite area of the mammal and the tick. Furthermore, this may reduce transfer of pathogens between the tick and the mammal or the extractor (i.e. the agent performing the removal process using the container or more generally the kit), and may cause the bite area B to heal more quickly.

A material of the container or bottle typically comprises glass or plastics material adapted to substantially resist alcoholic liquids.

When the container has the following dimensions, the prescribed volume of liquid disinfectant is preferably about 15 mL: neck has an inner diameter of ⅜ inches and height/length of 1.5 to 2 inches; body has a height of 1 to 1.5 inches.

As described hereinbefore, and according to one aspect, the present invention relates to a kit comprising a container with an elongated neck and reservoir and a method for removing a tick from a mammal, and more particularly to such a kit and method comprising liquid disinfectant submerging an imbedded tick and chemically shocking the tick, encouraging it to release and subsequently sanitizing the tick and the surrounding epidermal tissue (bite area), resulting in less damage to the epidermal tissue and faster healing.

The scope of the claims should not be limited by the preferred embodiments set forth in the examples but should be given the broadest interpretation consistent with the specification as a whole.

1. A kit for removing an imbedded tick from epidermal tissue of a mammal comprising:
a container configured to receive a liquid, wherein the container has a body and a neck collectively defining an interior of the container for receiving the liquid, wherein the body has a bottom arranged for resting on a support surface, wherein the neck has a rim delimiting an opening for transferring the liquid between the interior of the container and an exterior thereof, wherein the container further includes a cap configured to be removably received on the neck to selectively close the opening to resist transfer of the liquid from the interior to the exterior;
wherein the rim is arranged to be sized to surround the tick;
a prescribed volume of liquid disinfectant received in the container, wherein the liquid disinfectant is adapted to sanitize the epidermal tissue and the tick;
wherein the prescribed volume of liquid disinfectant is less than a volume of the interior of the container so as to provide an air gap between the rim of the container and a top surface of the liquid disinfectant in the container, such that the liquid disinfectant can flow to fill the neck upon tilting the container.

2. The kit of claim 1 further including tweezers for mechanically removing the tick.

3. The kit of claim 2 wherein the tweezers are configured to be received in the container in a storage position of the tweezers.

4. The kit of claim 3 wherein the tweezers are attached to the cap so as to be disposed in the storage position when the cap is received on the neck of the container.

5. The kit of claim 1 wherein, when the container extends between the bottom and the rim along an axis, the body of the container is enlarged relative to the neck in a radial direction of the axis and the neck is elongated relative to an axial direction of said axis.

6. The kit of claim 5 wherein the body of the container is enlarged relative to the neck non-symmetrically around the axis such that the axis is offset from a center of the body.

7. The kit of claim 5 wherein the neck has a height in the axial direction that is at least one-third of a height of the body in the axial direction.

8. The kit of claim 7 wherein the height of the neck is at least equal to a height of the body in the axial direction.

9. The kit of claim 1 wherein the liquid disinfectant is alcoholic and has an alcohol content of at least 70% by volume.

10. The kit of claim 9 wherein the alcohol content is at least 90% by volume.

11. The kit of claim 10 wherein the alcohol content is 99% by volume.

12. The kit of claim 1 wherein the rim has an inner diameter of about one-quarter inches to about one-half inches.

13. The kit of claim 12 wherein the inner diameter of the rim is about ⅜ inches.

14. The kit of claim 1 wherein the prescribed volume of the liquid disinfectant is greater than a volume of the neck of the container.

15. A method for extracting an imbedded tick from epidermal tissue of a mammal comprising:
providing a container with a prescribed volume of liquid disinfectant therein, wherein the prescribed volume of the liquid disinfectant is less than a volume of the container;
with the liquid disinfectant received in the container, locating a rim of the container on the mammal in a manner forming a seal therewith and surrounding the imbedded tick;
submerging the imbedded tick in the liquid disinfectant received in the container for a prescribed duration; and
removing, from the mammal, the container with the liquid disinfectant substantially received therein and the tick.

16. The method of claim 15 wherein removing the tick from the mammal comprises mechanically removing the tick using a tool distinct from the container.

17. The method of claim 15 wherein the prescribed duration for submerging the imbedded tick in the liquid disinfectant is at least 30 seconds.
